(19) United States
(12) Patent Application Publication
(10) Pub. No.: US 2024/0360479 A1
(43) Pub. Date: Oct. 31, 2024

(54) METHOD OF MAKING GRAPHENE TRANSPARENT BY FERMENTATION

(71) Applicant: Zhi-Min Li, Taichung City (TW)

(72) Inventor: Zhi-Min Li, Taichung City (TW)

(21) Appl. No.: 18/140,097

(22) Filed: Apr. 27, 2023

(51) Int. Cl. C12P 3/00; A61K 33/44; A61K 35/741

(52) U.S. Cl. CPC C12P 3/00; A61K 33/44; A61K 35/741; A61K 2035/115

(57) ABSTRACT

A method of making graphene transparent by fermentation contains: a step of preparing prerequisites; a first step in which the graphene mother bacteria, edible powders, powdered sugars, and a warm water and mixed and heated to produce a fermented graphene lump; a second step in which fermented graphene lump is smashed and smoothed out to a holding tray, then the holding tray is dried to form graphene enzyme lump. The method further contains: a third step in which the graphene enzyme lump is refined to graphene enzyme powders by a stirrer; a fourth step in which re-fermenting the graphene enzyme powders of the third step by repeating the first step, the second step and the third step; a fifth step in which the re-fermented graphene powders are soaked to produce edible powders, and the warm water is removed; a step sixth in which a transparent graphene containing probiotics is produced.

### TECHNICAL FIELD

The present disclosure relates to a method of making graphene transparent by fermentation which is configured to produce transparent graphene containing probiotics.

### BACKGROUND

Conventionally, graphene is a single-layer two-dimensional crystal composed of carbon atoms arranged in a hexagonal honeycomb crystal lattice with sp2 hybrid orbitals. The structure of graphene is very stable, and this stable lattice structure makes graphene have excellent thermal conductivity. Scientists have discovered that graphene oxide is super effective at inhibiting the growth of E. coli without harming human cells. Graphene oxide is also antibacterial to other bacteria, and graphene-derived compounds are thought to have applications in the treatment of cancer. The principle is that graphene oxide can recognize the difference in electron density between cancer cells and normal cells, and then attach to cancer stem cells, so that it can be acted on by targeted drugs and achieve the effect of inhibiting distant metastasis of tumors. This breakthrough discovery can make up for the shortcomings of traditional chemotherapy and radiotherapy that can only kill differentiated cancer cells, and is expected to achieve a higher treatment response rate and patient survival rate. At present, the team has experimentally proved that graphene oxide can inhibit the formation of tumor spheres in six cancers (breast cancer, pancreatic cancer, brain cancer, lung cancer, ovarian cancer, and prostate cancer), thereby inhibiting their spread

The present invention has arisen to mitigate and/or obviate the afore-described disadvantages.

### SUMMARY

A primary aspect of the present invention is to provide a method of making graphene transparent by fermentation which has antibacterial to relieve human discomfort.

To obtain above-mentioned aspect, a method of making graphene transparent by fermentation provided by the present invention contains:
a step of preparing prerequisites, wherein heating raw materials configured to produce graphene two times to produce graphene powders, and the graphene powders are mixed with yeast and warm waters and are placed in a temperature control room for multiple days to form graphene mother bacteria;
a first step in which the graphene mother bacteria are mixed with edible powders, powdered sugars, and a warm water in a container and are stirred and placed in a temperature control room, thereafter a temperature of the temperature control room is increased to a predetermined temperature so as to produce a fermented graphene lump;
a second step in which fermented graphene lump is smashed by a smashing tool and is poured and smoothed out to a holding tray, then the holding tray is put into the temperature control room to be dried for multiple days, thus forming graphene enzyme lump;
a third step in which the graphene enzyme lump is put into a stirrer to refine the graphene enzyme lump into graphene enzyme powders;
a fourth step in which re-fermenting the graphene enzyme powders of the third step by repeating the first step, the second step and the third step to produce re-fermented graphene powders;
a fifth step in which the re-fermented graphene powders are soaked in warm water for one day to produce edible powders, wherein a remained part of the warm water is removed after the edible powders deposit; and a step sixth in which a finished product is produced, wherein the finished product is the transparent graphene containing probiotics.

Preferably, in the step of preparing prerequisites, the raw materials are Phyllostachys edulis, the raw materials are heated to a temperature of 800° C. and are heated again to a temperature of 1000° C., and the graphene mother bacteria are formed for two days.

Preferably, in the first step, the temperature of the temperature control room is increased to 38° C. to 40° C.

Preferably, the graphene powders in the first step are fermented for three days.

Preferably, in the first step, the container is a plastic bucket or a glass bucket.

Preferably, in the second step, the holding tray is made of stainless steel.

Preferably, in the second step, the holding tray is dried for three days to four days.

Preferably, in the third step, a color of the graphene enzyme powders is gray black.

Preferably, in in the fourth step, a color of the re-fermented graphene powders is white.

Preferably, in in the fifth step a ratio of the re-fermented graphene powders and the warm water is 1:500 ratio.

Other objects and features will be in part apparent and in part pointed out hereinafter.

### BRIEF DESCRIPTION OF THE DRAWINGS

FIG. 1 is a flow chart of a method of making graphene transparent by fermentation according to a preferred embodiment of the present invention.

FIG. 2 is a block diagram showing a mixing ratio of graphene fermenting powders according to the preferred embodiment of the present invention.

FIG. 3 is a schematic view showing the operation of the fifth step and the sixth step according to the preferred embodiment of the present invention.

### DETAILED DESCRIPTION

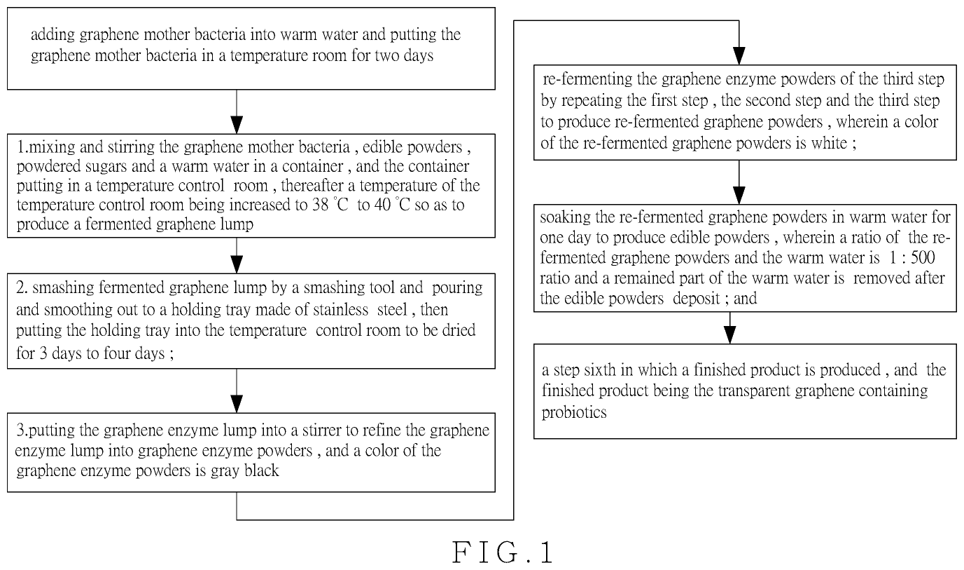

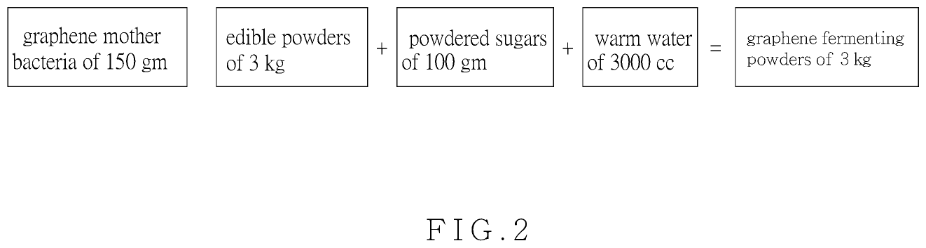

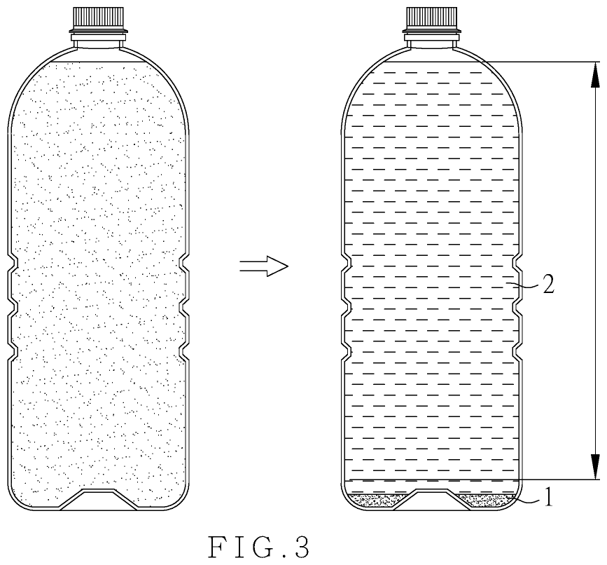

With reference to FIG. 1, a method of making graphene transparent by fermentation according to a preferred embodiment of the present invention comprises:
a step of preparing prerequisites, wherein heating raw materials configured to produce graphene to a temperature of 800° C., wherein the raw materials are Phyllostachys edulis, and the raw materials are heated again to a temperature of 1000° C. and are cooled to produce the graphene powders, thereafter the graphene powders are mixed with yeast and warm waters and placed in a room temperature or a temperature control room for two days to form graphene mother bacteria;
a first step in which the graphene mother bacteria are mixed with edible powders (such as sweet potato flours, corn flours or other edible powders), powdered sugars, and a warm water of a temperature of 30° C. (as shown in FIG. 2, a mixing ratio is 150 grams of graphene mother bacteria of 150 gm, edible powders of 3 kg, powdered sugars of 100 μm, and warm water of 3000 cc) to produce graphene fermenting powders of 3 kg, then the graphene fermenting powders of 3 kg are poured into a food container (such as a plastic bucket, a glass bucket or other food container) and are stirred and placed in the temperature control room, thereafter a temperature of the temperature control room is adjusted to a predetermined temperature (such as 38° C. to 40° C.), such that the graphene powders of 3 kg is fermented for three days to produce a fermented graphene lump;
a second step in which the fermented graphene lump is smashed by a smashing tool and is poured and smoothed out to a holding tray made of stainless steel, then the holding tray is put into the temperature control room to dried for three days to four days, thus forming graphene enzyme lump;
a third step in which the graphene enzyme lump is put into a stirrer to refine the graphene enzyme lump into graphene enzyme powders, and the graphene enzyme powders are put into the food container (such as the plastic bucket, the glass bucket or other food container), wherein a color of the graphene enzyme powders is gray black;
a fourth step in which re-fermenting the graphene enzyme powders of the third step by repeating the step 1 to step 3 to produce re-fermented graphene powders, wherein a color of the re-fermented graphene powders is white;
a fifth step in which the re-fermented graphene powders are soaked in 1:500 ratio of warm water for one day to produce edible powders 1, wherein a remained part of 1:500 ratio of warm (as shown in FIG. 3) water are removed after the edible powders 1 deposit;
a step sixth in which a finished product 2 (i.e. transparent graphene containing probiotics) is produced.

Thereby, the finished produced 2 does not damage human cells and has antibacterial to relieve human discomfort.

Having described the invention in detail, it will be apparent that modifications and variations are possible without departing from the scope of the invention defined in the appended claims.

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of making graphene transparent by fermentation comprising:
a step of preparing prerequisites, wherein heating raw materials configured to produce graphene two times to produce graphene powders, and the graphene powders are mixed with yeast and warm waters and are placed in a temperature control room for multiple days to form graphene mother bacteria;
a first step in which the graphene mother bacteria are mixed with edible powders, powdered sugars, and a warm water in a container and are stirred and placed in a temperature control room, thereafter a temperature of the temperature control room is increased to a predetermined temperature so as to produce a fermented graphene lump;
a second step in which fermented graphene lump is smashed by a smashing tool and is poured and smoothed out to a holding tray, then the holding tray is put into the temperature control room to be dried for multiple days, thus forming graphene enzyme lump;
a third step in which the graphene enzyme lump is put into a stirrer to refine the graphene enzyme lump into graphene enzyme powders;
a fourth step in which re-fermenting the graphene enzyme powders of the third step by repeating the first step, the second step and the third step to produce re-fermented graphene powders;
a fifth step in which the re-fermented graphene powders are soaked in warm water for one day to produce edible powders, wherein a remained part of the warm water is removed after the edible powders deposit; and
a step sixth in which a finished product is produced, and the finished product being the transparent graphene containing probiotics.

2. The method as claimed in claim 1, wherein in the step of preparing prerequisites, the raw materials are Phyllostachys edulis, the raw materials are heated to a temperature of 800° C. and are heated again to a temperature of 1000° C., and the graphene mother bacteria are formed for two days.

3. The method as claimed in claim 1, wherein in the first step, the temperature of the temperature control room is increased to 38° C. to 40° C.

4. The method as claimed in claim 1, wherein the graphene powders in the first step are fermented for three days.

5. The method as claimed in claim 1, wherein in the first step, the container is a plastic bucket or a glass bucket.

6. The method as claimed in claim 1, wherein in the second step, the holding tray is made of stainless steel.

7. The method as claimed in claim 1, wherein in the second step, the holding tray is dried for three days to four days.

8. The method as claimed in claim 1, wherein in the third step, a color of the graphene enzyme powders is gray black.

9. The method as claimed in claim 1, wherein in the fourth step, a color of the re-fermented graphene powders is white.

10. The method as claimed in claim 1, wherein in the fifth step a ratio of the re-fermented graphene powders and the warm water is 1:500 ratio.
